SEQUENCE LISTING (12 sequences)

SEQ ID NO: 1 - DNA, 25 bp, Artificial Sequence
  Primer
cggccgcggtcatagctgtttcctg

SEQ ID NO: 2 - DNA, 20 bp, Artificial Sequence
  Primer
cgccgccgccctccttgatg

SEQ ID NO: 3 - DNA, 25 bp, Artificial Sequence
  Primer
tctctgactgtggcgctccgttttg

SEQ ID NO: 4 - DNA, 26 bp, Artificial Sequence
  Primer
accagttccgttccagccttcttgat

SEQ ID NO: 5 - DNA, 22 bp, Artificial Sequence
  Primer
agccctaaattcaagccctgtg

SEQ ID NO: 6 - DNA, 20 bp, Artificial Sequence
  Primer
ggaaaccctggactactgcg

SEQ ID NO: 7 - DNA, 24 bp, Artificial Sequence
  Primer
aggtgagaaacaggcagaaatagt

SEQ ID NO: 8 - DNA, 25 bp, Artificial Sequence
  Primer
tgtccaaactcatcaatgtatctta

SEQ ID NO: 9 - DNA, 24 bp, Artificial Sequence
  Primer
gctttatggttcccttacggttta

SEQ ID NO: 10 - DNA, 24 bp, Artificial Sequence
  Primer
attatgggagagttggagccttca

SEQ ID NO: 11 - DNA, 23 bp, Artificial Sequence
  Primer
atacaccaacctagcccatcatt

SEQ ID NO: 12 - DNA, 22 bp, Artificial Sequence
  Primer
gacttaacctgcttcaccacaa

(19) United States
(12) Patent Application Publication
(10) Pub. No.: US 2017/0081679 A1
(43) Pub. Date: Mar. 23, 2017

(54) METHODS OF IMPROVING NUCLEASE MEDIATED HOMOLOGOUS RECOMBINATION

(71) Applicants: Jie Xu, Fairfax, VA (US); Yuqing Chen, Superior Township, MI (US); Jifeng Zhang, Ann Arbor, MI (US); Dongshan Yang, Ann Arbor, MI (US); Jun Song, Ann Arbor, MI (US)

(72) Inventors: Jie Xu, Fairfax, VA (US); Yuqing Chen, Superior Township, MI (US); Jifeng Zhang, Ann Arbor, MI (US); Dongshan Yang, Ann Arbor, MI (US); Jun Song, Ann Arbor, MI (US)

(21) Appl. No.: 15/265,291

(22) Filed: Sep. 14, 2016

Related U.S. Application Data
(60) Provisional application No. 62/284,200, filed on Sep. 22, 2015.

(51) Int. Cl. C12N 15/90; A61K 38/17; A01K 67/027; C12N 15/89; C12N 13/00

(52) U.S. Cl. CPC C12N 15/907; C12N 15/89; C12N 13/00; A01K 67/0275; A61K 38/1709; A61K 48/00

(57) ABSTRACT

The present invention relates to methods and compositions for improving nuclease mediated homologous recombination (HR). The invention is directly relevant to basic biomedical research, transgenic animal production, regenerative medicine, gene therapy, and other disciplines where HR is involved.

### CROSS-REFERENCE TO RELATED APPLICATION

This application claims priority under 35 U.S.C. §119(e) to U.S. Provisional Patent Application Ser. No. 62/284,200, filed on Sep. 22, 2015, the entire contents of which are hereby incorporated by reference.

### STATEMENT REGARDING FEDERALLY SPONSORED RESEARCH

The invention was made with government support under R01HL117491 and R01HL129778 awarded by National Institutes of Health. The government therefore has certain rights in the invention.

### FIELD OF PRESENT DISCLOSURE

This present disclosure relates to methods and compositions for improving nuclease mediated homologous recombination (HR).

### BACKGROUND INFORMATION

Zinc Finger Nuclease (ZFN), Transcription Activator-Like Effector Nuclease (TALEN), and CRISPR (clustered regularly interspaced short palindromic repeats)/Cas9 (CRISPR associated protein 9) are becoming major tools for genome editing. Importantly, knock-in in several non-rodent species has been finally achieved thanks to these customizable nucleases; yet the rates remain to be further improved.

Two major mechanisms, namely non-homologous end joining (NHEJ) and enhancing homology directed repair (HDR), function to repair DSBs. As its name suggests, in NHEJ, the break ends are directly ligated without the need for a homologous template, thus lead to generally unpredictable insertions or deletions (indels) at the targeting sites. HDR may take place, in addition to NHEJ, when homologous donor templates are present, leading to correct repair or knock-in events. We previously reported the production of KO rabbits using the ZFN and Cas9 system (references 1, 2; see the list of references at the end of the DETAILED DESCRIPTION OF THE INVENTION). The KO rates using the Cas9 system range from 10-100% in vitro, and 32.1-83.3% in vivo (reference 1). However, the frequency of HDR appears to be much lower than that of NHEJ. Without any intervention, the HDR/NHEJ ratio calculated by the number of indel events over that of knock-in events is below 10% in our rabbit system, consistent with reports in other species. For example, Gonzalez et al reported 2-3% HDR rates vs. 13-49% indel rates in human ES and iPS cells in 2014 (reference 3). Likewise, in one mouse study, the NHEJ mediated gene editing is 28-50%, whereas the HDR-mediated knock-in is below 10% (reference 4). Collectively, the HDR events take place at 1/3 or even lower rates than the NHEJ events.

Such low knock-in rate has become a bottleneck problem for the broad application of the Cas9 and other customizable nuclease systems in biomedical research, because for reliable disease modeling and gene correction it is often necessary that a specific change be introduced to the sequence. Even for gene addition therapy, it is desirable that such addition is location and copy number controlled, which has been demonstrated by knock-in to the ROSA26 or similar safe harbor locus.

### BRIEF SUMMARY OF THE INVENTION

The present invention is based on the discovery of that enhancing the HDR pathway is capable to dramatically improve nuclease mediated HR efficiencies in mammalian embryos. Specifically we describe novel methods for enhancing the HDR pathway for achieving the goal of nuclease mediated improving HR efficiencies.

Our in vitro results indicated that the application of an HDR enhancer, RS-1, increased the knock-in efficiency by 2- to 5-fold at different loci, including a ROSA26 like locus (RLL), Apolipoprotein A-1 (ApoAI), and cystic fibrosis transmembrane conductance regulator (CFTR), at its optimal concentration (7.5 μM); whereas NHEJ inhibitor SCR7 had minimal effects. We then applied RS-1 for animal production: one for Cas9 mediated knock-in of enhanced green fluorescence protein (EGFP) to RLL in the rabbit genome, and another one for TALEN mediated knock-in of human Apolipoprotein A-II (hApoAII) to the rabbit ApoAI (rbApoAI) locus. Consistent with in vitro results, we achieved multifold improvement on the knock-in rates for both. The efficiencies were 17.6 and 26.3% calculated as the ratio of total knock-in animals over total kits born with RS-1 supplementation, vs. 6.3 and 7.0% without RS-1 supplementation, respectively. Pronuclear microinjection of human RAD51 mRNA mimicked the beneficial results of RS-1 supplementation. The invention presents new tools to nuclease mediated knock-in animal production, and gene targeting in mammalian cells.

### BRIEF DESCRIPTION OF THE DRAWINGS

In the drawings:

FIG. 1 shows the effects of SCR7 (a NHEJ inhibitor) and RS-1 (a HDR enhancer) on Cas9 mediated knock-in rates in vitro. FIG. 1A shows efficiency of Cas9 mediated RLL-EGFP knock-in in rabbit embryos: Left panel shows effects of SCR7, and right panel shows effects of RS-1 at 0 μM, 7.5 μM and 15 μM, as well as injected RAD51 mRNA. FIG. 1B shows effects of optimized RS-1 treatment (at 7.5 μM) on knock-in to CFTR and ApoAI loci in rabbit embryos vs. control group (0 μM RS-1): Left panel shows Cas9 mediated knock-in of CFTRdelF508 mutation to rabbit CFTR, and right panel shows TALEN mediated knock-in of hApoAII to rbApoAI. *P<0.1. **P<0.05. NS: not significantly different.

FIG. 2 shows the effects of RS-1 on TALEN or Cas9 mediated knock-in rates in vivo. FIG. 2A shows the summary of knock-in animal production. KI: knock-in. Comparisons were made between RS-1 supplemented group (+) and non-treatment group (−) of the same locus. FIG. 2B depicts the gene targeting strategy of TALEN mediated knock-in of hApoAII to rbApoAI locus. FIG. 2C shows confirmation of hApoAII knock-in rabbits. Lane 1-10: samples from individual kits. Upper: PCR products using primer set LF1/LR1. Lower: PCR products using primer set RF1/RR1. M: molecule weight marker. Arrows indicate knock-in band. +: positive knock-in. −: negative knock-in. Kits #1, 7 and 10 are identified as knock-in. FIG. 2D shows the three hApoAII knock-in founder rabbits (DOB: Dec. 5, 2014). FIG. 2E depicts the gene targeting strategy of Cas9 mediated knock-in of EGFP to RLL locus. FIG. 2F shows confirmation of RLL-EGFP knock-in rabbits. Lane 1-13: samples from individual kits. Upper: PCR products using primer set LF2/LR2. Lower. PCR products using primer set RF2/RR2. M: molecule weight marker. Arrows indicate knock-in band. +: positive knock-in. −: negative knock-in. Kits#1, 2, 4, 9, 12 and 13 are identified as knock-in. FIG. 2G shows the three RLL-EGFP knock-in founder rabbits (DOB: Jun. 7, 2014). FIG. 2H shows the summary of germline transmission and allele distribution in embryos produced by RLL-EGFP founders. FIG. 2I shows the comparison of frequency of WT, indel and knock-in alleles between embryos parented by founders of the RS-1 treatment group and the non-treated group. *P<0.1. **P<0.05.

FIG. 3 shows the effects of SCR7 and RS-1 on embryo development and frequency of indel only embryos. FIG. 3A shows the embryo development rates. FIG. 3B shows the frequency of embryos containing indel only mutations. **P<0.05.

FIG. 4 shows the knock-in of CFTRdelF508 mutation to rabbit genome. FIG. 4A shows the gene targeting strategy of Cas9 mediated knock-in of CFTRdelF508 to CFTR locus in rabbit genome. FIG. 4B is a representative sequencing result showing successful knock-in of the CFTRdelF508 mutation. LF3, LR3, RF3, RR3 are primers used for PCR. Sequences of these primers as well as primers in other drawings are listed in the section below.

FIG. 5 shows the validation of the PCR assays using single primers. Left panel shows the PCR results by using primers pairs (RF1+RR1, or LF1+LR1) or single primers (LF1, LR1, RF2 or RR2) on an ApoAII knock-in founder. KI: knock-in. M: molecular weight marker. Knock-in specific bands were detected in both primer pairs (in triplicates), but not in single primer lanes. A non-specific band was present in the RR2 lane. Right panel shows the PCR results by using primers pairs (RF1+RR1, or LF1+LR1) or single primers (LF1, LR1, RF2 or RR2) on an RLL-EGFP knock-in founder. KI: knock-in. M: molecular weight marker. Knock-in specific bands were detected in both primer pairs, but not in single primer lanes.

### BRIEF DESCRIPTION OF THE SEQUENCES

Primers used for the experiments and referenced in the drawings are listed below. The corresponding SEQUENCE LISTING is submitted herewith via the EFS-Web.

Primers used for hApoAII knock-in experiment

Primers used for hApoAII knock-in experiment
SEQ ID 1 (LF1):
5′-CGGCCGCGGTCATAGCTGTTTCCTG-3′
SEQ ID 2 (LR1):
5′-CGCCGCCGCCCTCCTTGATG-3′
SEQ ID 3 (RF1):
5′-TCTCTGACTGTGGCGCTCCGTTTTG-3′
SEQ ID 4 (RR1):
5′-ACCAGTTCCGTTCCAGCCTTCTTGAT-3′
Primers used for RLL-EGFP knock-in experiment
SEQ ID 5 (LF2):
5′-AGCCCTAAATTCAAGCCCTGTG-3′
SEQ ID 6 (LR2):
5′-GGAAACCCTGGACTACTGCG-3′
SEQ ID 7 (RF2):
5′-AGGTGAGAAACAGGCAGAAATAGT-3′
SEQ ID 8 (RR2):
5′-TGTCCAAACTCATCAATGTATCTTA-3′
Primers used for CFTRdelF508 knock-in experiment
SEQ ID 9 (LF3):
5′-GCTTTATGGTTCCCTTACGGTTTA-3′
SEQ ID 10 (LR3):
5′-ATTATGGGAGAGTTGGAGCCTTCA-3′
SEQ ID 11 (RF3):
5′-ATACACCAACCTAGCCCATCATT-3′
SEQ ID 12 (RR3):
5′-GACTTAACCTGCTTCACCACAA-3′

### DETAILED DESCRIPTION OF THE INVENTION

The advent of ZFN, TALEN, and CRISPR/Cas9 technologies has changed the landscape of gene targeting. These customizable nucleases are efficient in generating double-strand breaks (DSB) in the genome that can lead to a functional knock-out (KO) of the targeted gene or be used to knock-in a DNA sequence at a specific locus in the genome in a number of species (references 5, 6). In 2012, we produced Apolipoprotein C3 (ApoCIII) KO rabbits using the ZFN approach (reference 2). In 2013, we successfully generated a number of KO rabbit lines using the Cas9 approach with high efficiencies (reference 1).

It is noted however, that the efficiency of knock-in animal production, even with the help of these nucleases, remains low. Cui et al reported in 2011 that the success rates of ZFN mediated knock-in in mouse and rat embryos after pronuclear stage microinjection range from 0.3-2.2% (reference 7). When TALEN was used in combination with oligodeoxynucleotides for microinjection to mouse embryos, the knock-in rate was 6.8% (1 knock-in founder out of 15 pups)(reference 8). Approximately 15% pups contain knock-in alleles when Cas9 and donor DNAs were microinjected to mouse embryos by the Jacnisch group (references 4, 9). Our experience with the rabbit models confirmed these findings: the knock-in rates are below 1% when calculated as the ratio of total knock-in kits over total embryos transferred, or 0-10% when calculated by ratio of total knock-in kits over total kits born. This low efficiency has become a rate limiting factor for a broader application of nuclease mediated gene modifications for transgenic animal production as well as in pluripotent stem cells.

Non-homologous end joining (NHEJ) and homology directed repair (HDR) are the two main mechanisms responsible for DNA repair after nucleases generate DSB at the target site (reference 10), where NHEJ would lead to KO characterized by unpredictable insertions or deletions (indels) whereas HDR results in knock-in events, when a donor vector is co-introduced. In the present invention, we examined the effects of a potent NHEJ inhibitor, SCR7 (reference 11), and an HDR enhancer, RS-1 (reference 12) on improving the efficiency of Cas9 or TALEN mediated knock-in in rabbits. We show that RS-1 enhances Cas9 and TALEN mediated knock-in efficiency in rabbit embryos both in vitro and in vivo. We also describe the beneficial effects of RAD51 on improving nuclease mediated knock-in rates.

### Example 1

### RS-1 Improves Nuclease Mediated Knock-in Rates in Mammalian Cells

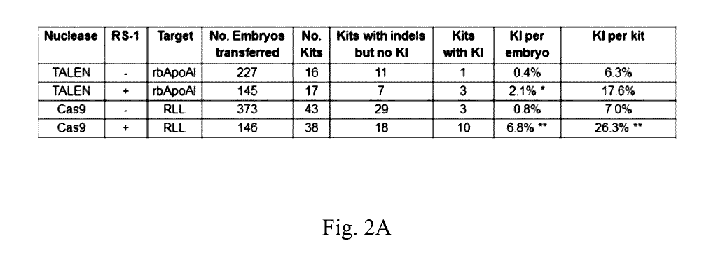

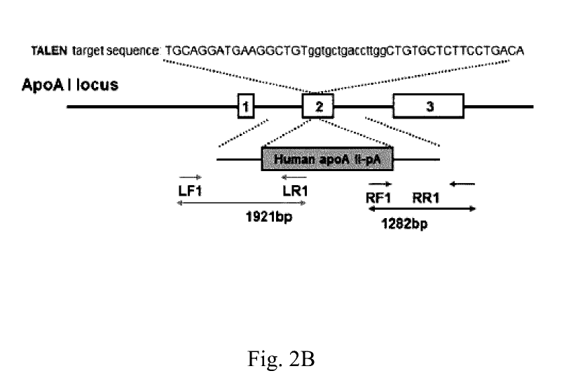

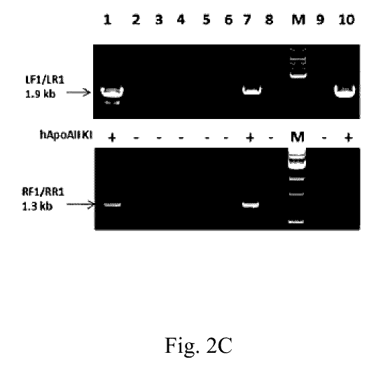

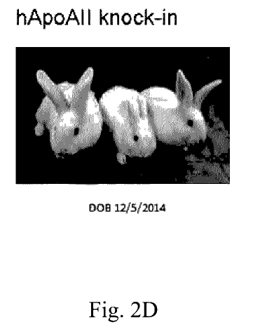

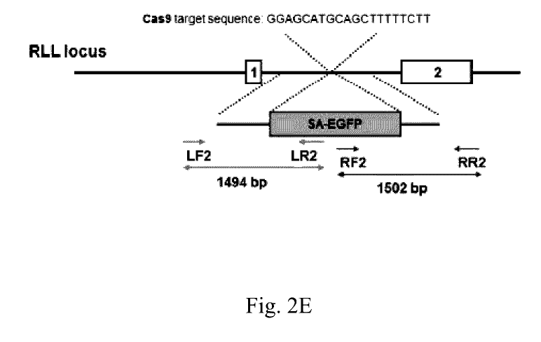

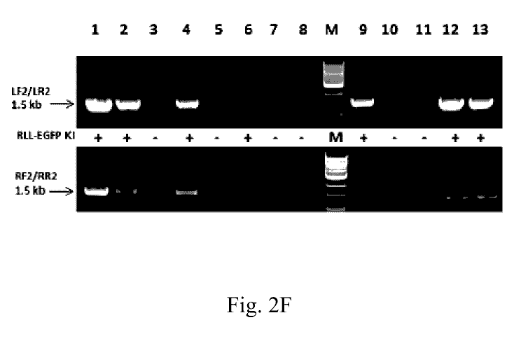

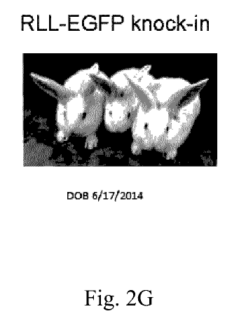

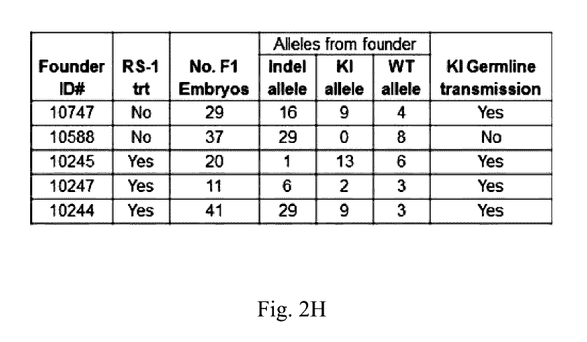

In one embodiment, we designed sgRNA targeting a ROSA26 like locus (RLL) in the rabbit genome (FIG. 2E). We next determined the effects of SCR7, an NHEJ inhibitor, and RS-1, an HDR enhancer on the knock-in success rates. Donor template DNAs were co-microinjected with Cas9 mRNA and sgRNA for RLL locus. After injection, the embryos were treated for 20 h in a serial concentration of SCR7: 0 μM (control), 20 μM, 40 μM, and 80 μM, or in RS-1 at 0 μM (control), 7.5 μM, and 15 μM.

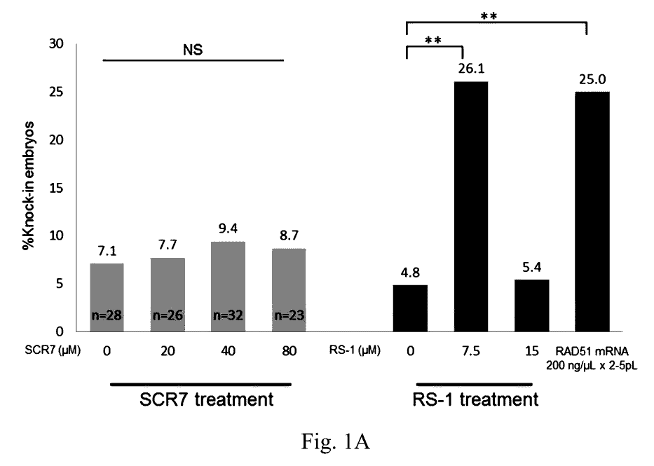

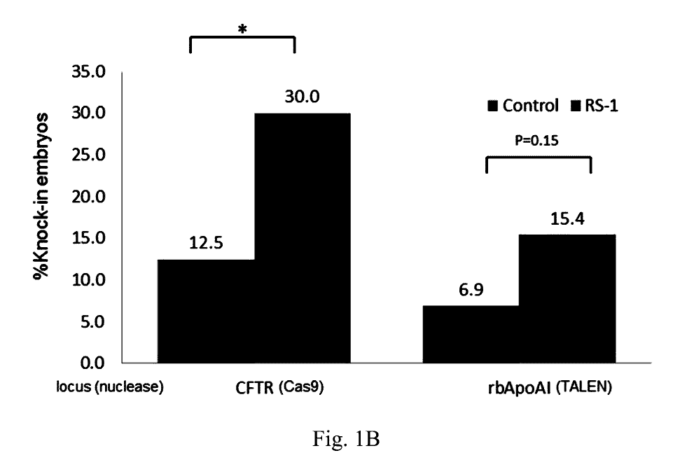

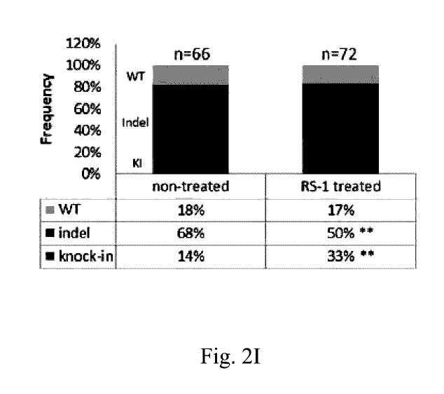

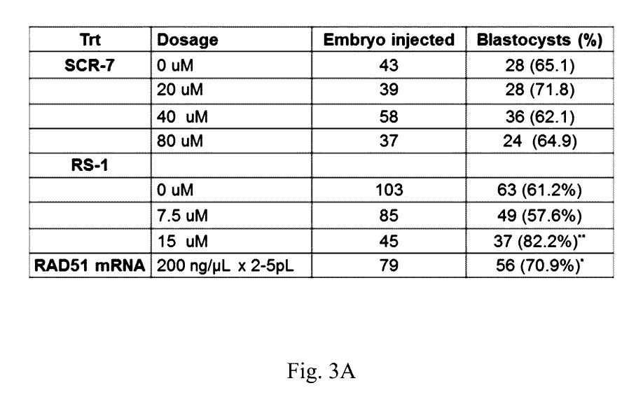

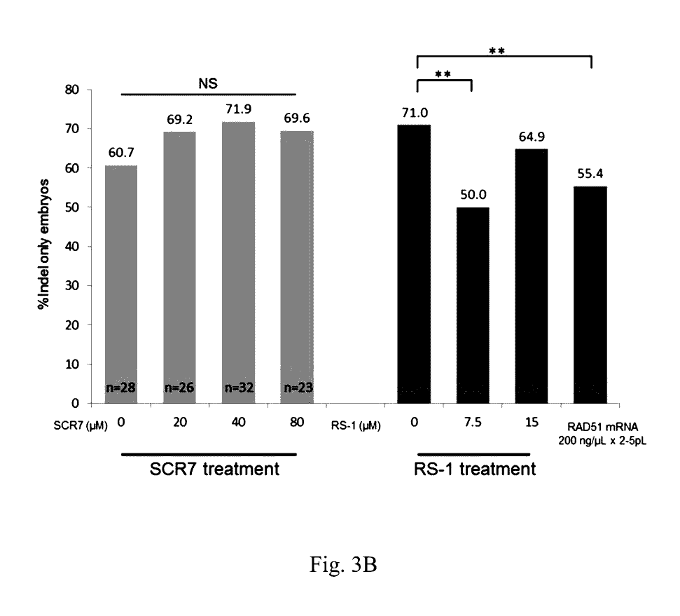

Treating embryos with SCR7 at these conditions had no effects on embryo development, as judged by blastocyst rates (62-72%, FIG. 3A). However, such treatments also have no significant effects on the overall knock-in efficiency (7.1% in the control vs. 7.7-9.4% in the treatment groups). (FIG. 1A)

Treating embryos with RS-1 at 15 μM appears to enhance the blastocyst development (82.2 vs. 61.2% in the control, FIG. 3); but the knock-in efficiency was not improved (5.4 vs. 4.8% in the control, FIG. 1A). Treating embryos with RS-1 at 7.5 μM, surprisingly, resulted in 26.1% (12 out of 46) knock-in rate, significantly higher than those of control (4.8%) and at 15 μM (5.4%). The blastocyst development at this condition was 57.6%, similar to that of the control group.

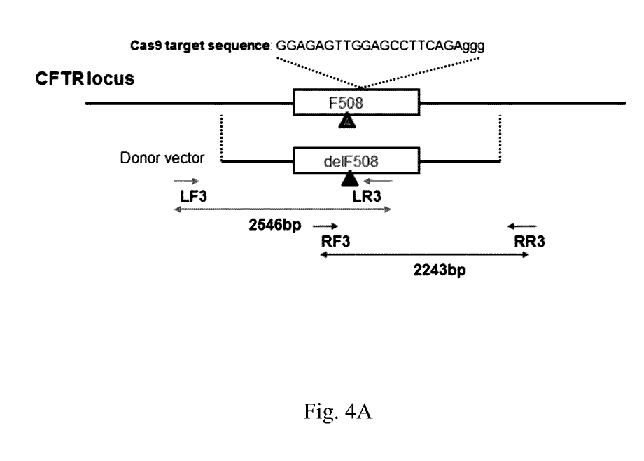

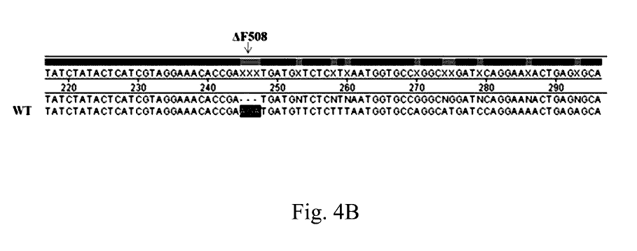

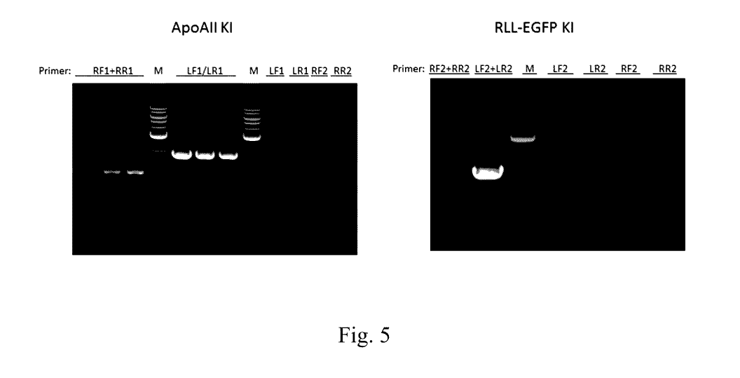

In another embodiment, we tested the effects of RS-1 supplementation on Cas9 mediated knock-in of CFTRdelF508, the most frequent mutation type identified in human cystic fibrosis (CF) patients, to the rabbit CFTR locus (FIG. 4).

In another embodiment, we tested the effects of RS-1 in a TALEN mediated knock-in system. We designed and validated TALEN pairs targeting rbApoAI locus for knocking in the hApoAII coding sequence (FIG. 2B).

Consistently, higher percentage of embryos carrying knock-in alleles were obtained in both the CFTRdelF508 (30 vs. 13%) and hApoAII (15 vs. 7%) cases when RS-1 supplementation was employed. These data indicate that RS-1 treatment work on different loci, and with different types of customizable nucleases.

### Example 2

### RS-1 Improves Nuclease Mediated Knock-in Rates for Knock-in Animal Production

In one embodiment, we used RS-1 at 7.5 μM for the in vivo experiments. The sgRNA and donor DNA were used for knocking in EGFP to the rabbit RLL.

In the control group, we transferred a total of 373 embryos to synchronized recipient rabbits and obtained 43 kits. Twenty-nine of these 43 kits (67%) carried indel alleles but with on knock-in alleles. Three are proven as knock-in founders after PCR and sequencing (FIG. 2A). Of note, these three knock-in founders also carry indel alleles. The overall knock-in efficiency is 0.8% calculated by total knock-in founders/total embryos transferred, or 7.0% calculated by total knock-in founders/total kits born.

In the RS-1 treatment group (FIG. 2A), we transferred 146 embryos and obtained 38 kits, 18 of which carried indel but no knock-in alleles (47%). Similar to the in vitro results, the knock-in success rate was multifold higher than that in the control group. A total of 10 knock-in founders were produced, with the knock-in efficiency of 6.8% calculated by total knock-in founders/total embryos transferred, or 26.3% calculated by total knock-in founders/total kits born.

In another embodiment, we tested RS-1 on the TALEN system in vivo. The same TALEN pairs and donor DNAs used in the in vitro work were used to product hApoAII knock-in animals.

Without RS-1 treatment, only one founder animal was produced out of 227 embryos transferred (FIG. 2A). The knock-in efficiency was 0.4% calculated by total knock-in founders/total embryos transferred or 6.3% calculated by total knock-in founders/total kits born.

After RS-1 treatment, we transferred a total of 145 embryos and obtained 17 kits. Seven out of 17 kits carried indel but no knock-in alleles (41%). Three rabbits were confirmed as knock-in founders (FIG. 2A), resulting in the knock-in efficiency of 2.1% calculated by total knock-in founders/total embryos transferred or 17.6% calculated by total knock-in founders/total kits born), multifold higher than the control group.

No abnormalities are found in both the RLL-EGFP and hApoAII founder animals derived from both the RS-1 treatment and control groups (FIGS. 2D & G). At the time of submitting this manuscript, five RLL-EGFP founders, three from the RS-1 treatment group, two from the non-treatment group, have reached sexual maturity. We bred these RLL-EGFP founder animals with WT counterparts.

Sixty-six embryos parented by the founders of the non-treatment group were collected. Nine embryos carried the knock-in allele (14%); however, all came from the same founder (#10747). The knock-in alleles in the other founder (#10588) appears not germline transmitting.

Seventy-two embryos parented by the founders of the RS-1 treatment group were collected. Twenty-four embryos carried the knock-in allele (33%). Notably, all three founders (#10245, 10247, and 10244) in this group germline transmitted the knock-in allele.

We also allowed one pregnancy fathered by founder animal #10245 to full term, resulted in 8 kits, 4 of which (50%) carry the RLL-EGFP knock-in genotyping, and as expected, all expressed EGFP consecutively.

To ultimately determine the frequencies of different types of alleles (i.e. WT, indel and knock-in) in the germ cells of knock-in animals, we used WT animals to breed with five RLL knock-in founders, 2 derived without RS-1 treatment and the other 3 with RS-1 treatment. Embryos were produced from founders of the RS-1 treated group (n=72) and the non-treated group (n=66), sequenced, and categorized as WT, indel, or knock-in based on the allele sequence (FIG. 2I). Similar percentage of WT alleles (17 vs. 18%) are found between these two groups. Notably, consistent with the findings at the whole organism level, the knock-in frequency at the allele level was significantly higher in the RS-1 treated group (33%) than in the non-treated group (14%). It appears that knock-in events take place at the expense of KO because the overall mutation rates (indel+knock-in) were similar between these two groups. These data show that in addition to enhancing the chance of obtaining the knock-in founder animals, RS-1 treatment further increased the percentage of knock-in alleles in the derivative embryos/animals. These two factors multiply to result in the significantly improved efficiency of knock-in animal production.

In sum, RS-1 is effective on improving knock-in rates in both the TALEN and the Cas9 mediated genome editing systems. RS-1 treatment does not appear to have any toxic effects on the overall animal health and reproduction. All three animals generated from the RS-1 treatment group are germline transmitting (100%); whereas only one of the two from the non-treatment group is germline transmitting (50%), indicating that RS-1 does not adversely affect, if not improves, the germline transmitting capacity of the knock-in founder animals.

### Example 3

### Microiniection of RAD51 mRNA Mimics the Beneficial Effects of RS-1 Supplementation

In one embodiment, we microinjected 79 embryos with RAD51 mRNA, in addition to sgRNA, Cas9 mRNA and donor DNAs (FIG. 3A). RAD51 recombinase is a key player for homologous recombination and the repair of DNA DSBs (reference 13). Since it has been reported that RS-1 stimulates RAD51 (reference 12), we reasoned that including RAD51 mRNA in the microinjection mix would yield similar improvements on the knock-in efficiency as achieved with RS-1.

Fifty-six of these embryos (71%) developed to blastocyst stage, higher than those in the control group. All 56 blastocysts were PCR and sequenced, out of which 14 are knock-in positive (25%), significantly higher than that in the control group (4.8%).

These results suggest that co-microinjection of RAD51 may be used in substitution of RS-1 treatment to simplify the procedure. It also suggests that RS-1 likely functions through stimulating RAD51 to enhance the nuclease mediated knock-in efficiency.

Below is the list of references referenced herein.
1. Yang D, et al. (2014) Effective gene targeting in rabbits using RNA-guided Cas9 nucleases. J Mol Cell Biol 6(1):97-99.
2. Yang D, et al. (2013) Production of apolipoprotein C-III knockout rabbits using zinc finger nucleases. Journal of visualized experiments: JoVE (81):e50957.
3. Zhu Z, Gonzalez F, & Huangfu D (2014) The iCRISPR platform for rapid genome editing in human pluripotent stem cells. Methods in enzymology 546:215-250.
4. Wang H, et al. (2013) One-step generation of mice carrying mutations in multiple genes by CRISPR/Cas-mediated genome engineering. Cell 153(4):910-918.
5. Carlson D F, et al. (2012) Efficient TALEN-mediated gene knockout in livestock. Proceedings of the National Academy of Sciences of the United States of America.
6. Clark K J, Voytas D F, & Ekker S C (2011) A TALE of two nucleases: gene targeting for the masses? Zebrafish 8(3): 147-149.
7. Cui X, et al. (2011) Targeted integration in rat and mouse embryos with zinc-finger nucleases. Nature biotechnology 29(1):64-67.
8. Panda S K, et al. (2013) Highly efficient targeted mutagenesis in mice using TALENs. Genetics 195(3):703-713.
9. Yang H, et al. (2013) One-step generation of mice carrying reporter and conditional alleles by CRISPR/Cas-mediated genome engineering. Cell 154(6): 1370-1379.
10. Ran F A, et al. (2013) Genome engineering using the CRISPR-Cas9 system. Nature protocols 8(11):2281-2308.
11. Srivastava M, et al. (2012) An inhibitor of nonhomologous end-joining abrogates double-strand break repair and impedes cancer progression. Cell 151(7):1474-1487.
12. Jayathilaka K, et al. (2008) A chemical compound that stimulates the human homologous recombination protein RAD51. Proceedings of the National Academy of Sciences of the United States of America 105(41):15848-15853.
13. Klein H L (2008) The consequences of Rad51 overexpression for normal and tumor cells. DNA repair 7(5):686-693.

### Other Embodiments

Various other adaptations and combinations of features of the embodiments and implementations disclosed are within the scope of the present disclosure. It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating mammalian cells and embryos with a small molecule or a combination of small molecules for enhancing the nuclease mediated HR rates.

2. A method of introduction of transcriptional (e.g. mRNA) or translational products (e.g. protein) of HDR pathway components (e.g. RAD51) to the mammalian cells and embryos for enhancing nuclease mediated HR rates.

3. A method of claim 1 where the small molecule is RS-1.

4. A method of claim 1 where the small molecule is RS-1 mimics.

5. A method of claim 1 where the treatment involves supplementing RS-1 at 2.5 to 12.5 μM in the cell or embryo culture medium.

6. A method of claim 1 where the treatment involves supplementing RS-1 at 2.5 to 12.5 μM in the cell or embryo manipulation medium.

7. A method of claim 1 where the treatment involves supplementing RS-1 at 2.5 to 12.5 μM in the electroporation medium.

8. A method of claim 2 where the transcriptional products are mRNAs.

9. A method of claim 2 where the translational products are proteins.

10. A method of claim 2 where the transcriptional or translational products are microinjected to fertilized or unfertilized mammalian embryos.

11. A method of claim 2 where the transcriptional or translational products are electroporated.

12. A method of supplementing energy molecules to the process of claim 1 and claim 2.

13. A method of claim 12 where the energy molecule is adenosine triphosphate (ATP).

14. A method of claim 12 where the energy molecule is glucose.

15. A method of utilizing mammalian embryos or cells derived from method or method combinations of claim 1 to 14 for biomedical research.

16. A method of utilizing mammalian embryos or cells derived from method or method combinations of claim 1 to 14 for production of genetically modified animals.

17. A method of utilizing mammalian embryos or cells derived from method or method combinations of claim 1 to 14 for regenerative medicine therapeutic in humans and mammalian animal species.

18. A method of utilizing mammalian embryos or cells derived from method or method combinations of claim 1 to 14 for gene therapy in humans and mammalian animal species.
